(19) United States
(12) Patent Application Publication
(10) Pub. No.: US 2022/0125841 A1
(43) Pub. Date: Apr. 28, 2022

(54) SODIUM FLUORESCEIN AS A REVERSAL AGENT FOR AN ANTI-FLUORESCEIN CAR T CELLS AND FLUORESCEIN-PHOSPHOLIPID-ETHERS OR PROFLUORESCEIN-PHOSPHOLIPID-ETHERS

(71) Applicant: Seattle Children's Hospital (dba Seattle Children'a Research Institute), Seattle, WA (US)

(72) Inventors: Michael C. Jensen, Seattle, WA (US); James Matthaei, Seattle, WA (US)

(21) Appl. No.: 17/438,231

(22) PCT Filed: Mar. 11, 2020
(86) PCT No.: PCT/US2020/022130

§ 371 Date: Sep. 10, 2021

Related U.S. Application Data
(60) Provisional application No. 62/818,030, filed on Mar. 13, 2019.

(51) Int. Cl. A61K 35/17; A61P 35/00; C07K 14/725

(52) U.S. Cl. CPC A61K 35/17; C07K 14/7051; A61P 35/00

(57) ABSTRACT

Some embodiments of the methods and compositions provided herein relate to modulating signaling of anti-hapten CAR T cells, such as anti-fluorescein CAR T cells, by the use or administration of an unconjugated hapten, such as unconjugated fluorescein or a salt or derivative thereof.

### CROSS-REFERENCE TO RELATED APPLICATIONS

This application claims the benefit of U.S. Provisional Application No. 62/818,030 filed Mar. 13, 2019, hereby expressly incorporated by reference in its entirety.

### REFERENCE TO SEQUENCE LISTING

The present application is being filed along with a Sequence Listing in electronic format. The Sequence Listing is provided as a file entitled SCRI227WOSEQLIST.TXT, created Mar. 11, 2020, which is 14,018 bytes in size. The information in the electronic format of the Sequence Listing is hereby expressly incorporated by reference in its entirety.

### FIELD OF THE INVENTION

Some embodiments of the methods and compositions provided herein relate to modulating the signaling of anti-hapten CAR T cells, such as anti-fluorescein CAR T cells, by the use of or administration of an unconjugated hapten, such as unconjugated fluorescein or a derivative thereof, such as a salt thereof.

### BACKGROUND OF THE INVENTION

The adoptive transfer of human T lymphocytes that are engineered by gene transfer to express chimeric antigen receptors (CARs) specific for surface molecules expressed on tumor cells has the potential to effectively treat cancer. Chimeric receptors are synthetic receptors that include an extracellular ligand binding domain, most commonly a single chain variable fragment of a monoclonal antibody (scFv) linked to intracellular signaling components, most commonly CD3ζ alone or combined with one or more costimulatory domains. Much of the research in the design of chimeric antigen receptors has focused on defining scFvs and other ligand binding elements that target malignant cells without causing serious toxicity to essential normal tissues, and on defining the optimal composition of intracellular signaling modules to activate T cell effector functions. There remains a need for a CAR T cell-mediated therapy that is selective for specific targets and which minimizes adverse side effects.

### SUMMARY OF THE INVENTION

Some embodiments of the methods and compositions provided herein include a method of treating, ameliorating, inhibiting, or providing a therapy to a subject having a cancer, comprising, consisting essentially of, or consisting of: (a) administering to the subject a composition comprising, consisting essentially of, or consisting of a lipid conjugated to a target moiety; (b) administering a cell to the subject, wherein the cell comprises, consist essentially of, or consists of a chimeric antigen receptor (CAR) or T cell receptor (TCR), which specifically binds to the target moiety; and (c) administering the target moiety unconjugated to the lipid to the subject.

In some embodiments, step (c) is performed subsequent to step (b).

In some embodiments, administration of the unconjugated target moiety reduces effector function of the CAR T cell, compared to effector function of the CAR T cell in the absence of the unconjugated target moiety. In some embodiments, administration of the unconjugated target moiety reduces cytokine production from the CAR T cell compared to cytokine production from the CAR T cell in the absence of the unconjugated target moiety.

In some embodiments, the target moiety comprises, consists essentially of, or consists of biotin, digoxigenin, dinitrophenol, fluorescein, or a derivative or salt thereof. In some embodiments, the salt is a sodium, disodium, potassium, or dipotassium salt of biotin, digoxigenin, dinitrophenol, or fluorescein. In some embodiments, the salt is a calcium, magnesium, monophosphate, diphosphate, hydrochloride, sulfate, acetate, chloride, maleate, citrate, mesylate, nitrate, tartrate, aluminum, or gluconate salt. In some embodiments, the target moiety comprises, consists essentially of, or consists of fluorescein, or a derivative or salt thereof, such as sodium fluorescein.

In some embodiments, the target moiety is unconjugated. In some embodiments, the target moiety is conjugated. In some embodiments, the target moiety is conjugated to a nucleic acid, DNA, RNA, nucleotide, sugar, carbohydrate, peptide, polypeptide, protein, antibody, hormone, lipid, ether lipid, phospholipid, or cholesterol. In some embodiments, the target moiety is conjugated to a phospholipid ether.

In some embodiments, the lipid comprises, consists essentially of, or consists of a phospholipid ether (PLE).

In some embodiments, the cell is a precursor T cell. In some embodiments, the cell is a hematopoietic stem cell. In some embodiments, the cell is a CD8+T cytotoxic lymphocyte cell selected from the group consisting of naïve CD8+ T cells, central memory CD8+ T cells, effector memory CD8+ T cells and bulk CD8+ T cells. In some embodiments, the cell is a CD4+T helper lymphocyte cell that is selected from the group consisting of naïve CD4+ T cells, central memory CD4+ T cells, effector memory CD4+ T cells, and bulk CD4+ T cells.

In some embodiments, the cancer is a solid tumor, such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma.

In some embodiments, the subject is an animal. In some embodiments, the subject is a mammal. In some embodiments, the subject is a human, dog, cat, mouse, rat, cow, pig, horse, or chicken.

Embodiments provided herein are described by way of the following numbered alternatives:

1. A method of treating, ameliorating, inhibiting, or providing a therapy to a subject having a cancer, comprising:

(a) administering to the subject a composition comprising a lipid conjugated to a target moiety, such as biotin, digoxigenin, dinitrophenol or fluorescein;

(b) administering a cell to the subject, wherein the cell comprises a chimeric antigen receptor (CAR) or T cell receptor (TCR), which specifically binds to the target moiety; and

(c) administering the target moiety unconjugated to the lipid to the subject, such as a salt of biotin, digoxigenin, dinitrophenol or fluorescein, e.g., sodium, disodium, or a potassium salt of biotin, digoxigenin, dinitrophenol or fluorescein.

2. The method of alternative 1, wherein step (c) is performed subsequent to step (b).

3. The method of alternative 1 or 2, wherein administration of the unconjugated target moiety reduces effector function of the cell comprising the CAR or TCR, as compared to effector function of the cell comprising the CAR or TCR in the absence of the unconjugated target moiety.

4. The method of any one of alternatives 1-3, wherein administration of the unconjugated target moiety reduces cytokine production from the cell comprising the CAR or TCR, as compared to cytokine production from the cell comprising the CAR or TCR in the absence of the unconjugated target moiety.

5. The method of any one of alternatives 1-4, wherein the target moiety comprises biotin, digoxigenin, dinitrophenol, fluorescein, or a derivative thereof.

6. The method of any one of alternatives 1-5, wherein the target moiety comprises fluorescein, or derivative thereof.

7. The method of any one of alternatives 1-6, wherein the lipid comprises a phospholipid ether (PLE).

8. The method of any one of alternatives 1-7, wherein the cell is a precursor T cell.

9. The method of any one of alternatives 1-8, wherein the cell is a hematopoietic stem cell.

10. The method of any one of alternatives 1-9, wherein the cell is a CD8+T cytotoxic lymphocyte cell selected from the group consisting of naïve CD8+ T cells, central memory CD8+ T cells, effector memory CD8+ T cells and bulk CD8+ T cells.

11. The method of any one of alternatives 1-9, wherein the cell is a CD4+T helper lymphocyte cell that is selected from the group consisting of naïve CD4+ T cells, central memory CD4+ T cells, effector memory CD4+ T cells, and bulk CD4+ T cells.

12. The method of any one of alternatives 1-11, wherein the cancer is a solid tumor, such as a colon cancer, breast cancer, ovarian cancer, lung cancer, pancreatic cancer, prostate cancer, melanoma, renal cancer, pancreatic cancer, brain cancer, glioblastoma, neuroblastoma, medulloblastoma, sarcoma, bone cancer, or liver cancer, or a non-solid tumor, such as a leukemia, or a multiple myeloma.

13. The method of any one of alternatives 1-12, wherein the subject is a mammal.

14. The method of any one of alternatives 1-13, wherein the subject is human.

15. The method of any one of alternatives 1-14 wherein the target moiety unconjugated to the lipid is a salt of biotin, digoxigenin, dinitrophenol or fluorescein, e.g., sodium, disodium, or a potassium salt of biotin, digoxigenin, dinitrophenol or fluorescein.

16. The method of any one of alternatives 1-15, wherein the target moiety is fluorescein and the target moiety unconjugated to the lipid is a salt of fluorescein, preferably sodium or disodium fluorescein.

17. The method of any one of alternatives 1-16, wherein the CAR or TCR comprises a sequence selected from the group consisting of SEQ ID NOs: 1-6.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1A depicts a flow cytometry analysis for FL-PLE integration into cells.

FIG. 1B depicts a chromium release assay for effector T cells (CD8+ Mock or CD8+ antiFL(FITC-E2) CAR(Lg) T cells) mixed at various ratios with target cells (effector:target, E:T). The target cells are K562 control, K562 OKT3+, and K562 with 5 μM FL-PLE. One CD8+ antiFL(FITC-E2) population was exposed to 50 μM NaFL.

FIG. 1C depicts a cytokine release assay for helper cells (CD4+ Mock or CD4+ antiFL(FITC-E2) CAR(Lg) T cells) and effector T cells (CD8+ Mock or CD8+ antiFL(FITC-E2) CAR(Lg) T cells) mixed with target cells. The target cells are K562 control, K562 OKT3+, and K562 with 5 μM FL-PLE. One CD4+ and CD8+ antiFL(FITC-E2) population was exposed to 50 μM NaFL.

FIG. 2A depicts tumor progression in an MDA-MB-231 adenocarcinoma tumor xenograft mouse model as measured by total flux (photons/sec) over time. The tumor is bioluminescent with the addition of eGFP and luciferase genes.

FIG. 2B depicts levels of cytokine release syndrome (CRS) and cytotoxicity in an MDA-MB-231 adenocarcinoma tumor xenograft mouse model over time. FIG. 2B (lower panel) is an enlargement of an area of FIG. 2B (upper panel).

FIG. 2C depicts percent survival in an MDA-MB-231 adenocarcinoma tumor xenograft mouse model.

FIG. 3A depicts CD4+ and CD8+ ratios of a T cell population from two donors after lentiviral transduction of the antiFL(Mut2) CAR (or Mock control), selection with methotrexate, and expansion.

FIG. 3B depicts successful CAR positivity of a T cell population from two donors after lentiviral transduction of the antiFL(Mut2) CAR (or Mock control), selection with methotrexate, and expansion.

FIG. 3C depicts flow cytometry detection of NaFL (0 or 5 μM) in K562 parental (control), K562 OKT3+, and MDA-MB-231 adenocarcinoma target cells.

FIG. 3D depicts a cytokine release assay measuring IL-2 using the effector cells generated in FIG. 3A-B, target cells tested in FIG. 3C, and a dose titration of NaFL (0, 1, 5, or 10 μM). The effect of NaFL on reducing cytokine activity of antiFL(Mut2) CAR T cells is dose dependent.

FIG. 3E depicts a cytokine release assay measuring IFN-γ using the effector cells generated in FIG. 3A-B, target cells tested in FIG. 3C, and a dose titration of NaFL (0, 1, 5, or 10 μM). The effect of NaFL on reducing cytokine activity of antiFL(Mut2) CAR T cells is dose dependent.

FIG. 3F depicts a cytokine release assay measuring TNF-α using the effector cells generated in FIG. 3A-B, target cells tested in FIG. 3C, and a dose titration of NaFL (0, 1, 5, or 10 μM). The effect of NaFL on reducing cytokine activity of antiFL(Mut2) CAR T cells is dose dependent.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood by one of ordinary skill in the art to which the present disclosure belongs. For purposes of the present disclosure, the following terms are defined below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques within the skill of the art.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

As used herein, the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence.

As used herein, the term “antibody” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)2, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some embodiments, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains, and incorporate only the complementarity-determining regions of the animal-derived antibody. This can be accomplished by carefully examining the sequence of the hyper-variable loops of the variable regions of a monoclonal antigen binding unit or monoclonal antibody, and fitting them to the structure of a human antigen binding unit or human antibody chains.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In some embodiments, definitive delineation of a CDR and identification of residues comprising the binding site of an antibody is accomplished by solving the structure of the antibody and/or solving the structure of the antibody-ligand complex. In some embodiments, that can be accomplished by any of a variety of techniques known to those skilled in the art, such as X-ray crystallography. In some embodiments, various methods of analysis can be employed to identify or approximate the CDR regions. In some embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM.TM., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. In some embodiments containing more than one CDR, the CDRs may be defined in accordance with any of Kabat, Chothia, extended, IMGT, Paratome, AbM, and/or conformational definitions, or a combination of any of the foregoing. In some embodiments, the residue number of a variable region is numbered using the IMGT numbering system.

As known in the art, a “constant region” of an antibody refers to the constant region of the antibody light chain or the constant region of the antibody heavy chain, either alone or in combination.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration. By “co-administer” it is meant that a first compound described herein is administered at the same time, ju1st prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype (e.g., fibrosis or cancer). As used throughout, “modulation” is meant to refer to an increase or a decrease in the indicated phenomenon (e.g., modulation of a biological activity refers to an increase in a biological activity or a decrease in a biological activity).

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The term “immune cells” refers to cells of hematopoietic origin that are involved in the specific recognition of antigens. Immune cells include antigen presenting cells (APCs), such as dendritic cells or macrophages, B cells, T cells, natural killer cells, and myeloid cells, such as monocytes, macrophages, eosinophils, mast cells, basophils, and granulocytes.

The term “immune response” refers to T cell-mediated, NK cell-mediated, macrophage-mediated, and/or B cell-mediated immune responses. Exemplary immune responses include B cell responses (e.g., antibody production), NK cell responses or T cell responses (e.g., cytokine production, and cellular cytotoxicity) and activation of cytokine responsive cells, e.g., macrophages. The term “activating immune response” refers to enhancing the level of T-cell-mediated and/or B cell-mediated immune response, using methods known to one of skilled in the art. In some embodiments, the level of enhancement is at least 20-50%, alternatively at least 60%, at least 70%, at least 80%, at least 90%, at least 100%, at least 120%, at least 150%, or at least 200%.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, various types of wetting agents, detergents such as polysorbate 20 to prevent aggregation, and sugars such as sucrose as cryoprotectant. Preferred diluents for aerosol or parenteral administration are phosphate buffered saline (PBS) or normal (0.9%) saline.

As used herein, a “carrier” refers to a compound, particle, solid, semi-solid, liquid, or diluent that facilitates the passage, delivery and/or incorporation of a compound to cells, tissues and/or bodily organs. For example, without limitation, a lipid nanoparticle (LNP) is a type of carrier that can encapsulate an oligonucleotide to thereby protect the oligonucleotide from degradation during passage through the bloodstream and/or to facilitate delivery to a desired organ, such as to the liver.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “excipient” has its ordinary meaning as understood in light of the specification, and refers to inert substances, compounds, or materials added to a pharmaceutical composition to provide, without limitation, bulk, consistency, stability, binding ability, lubrication, disintegrating ability etc., to the composition. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. The amount of the excipient may be found in a pharmaceutical composition at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with an immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

Some embodiments of the methods and compositions provided herein relate to modulating signaling of anti-hapten CAR T cells, such as anti-fluorescein CAR T cells, by the use or administration of an unconjugated hapten, such as fluorescein or a derivative or salt thereof, for instance sodium fluorescein.

In some embodiments, anti-fluorescein CAR T cells can work in conjugation with bispecific compounds such as fluorescein conjugated with a phospholipid ether (FL-PLE) or precursor FL-PLE (ProFL-PLE) in cancer therapies. Both FL-PLE and ProFL-PLE include a tumor targeting moiety (PLE) and a CAR recognition/target moiety (FL) that are linked together via a spacer element. In some embodiments, the Pro moiety can provide an added level of safety that prevents recognition of the ProFL-PLE by the antiFL CAR T cell until it is processed in a tumor microenvironment to provide FL-PLE. In some embodiments, the FL-PLE has the structure

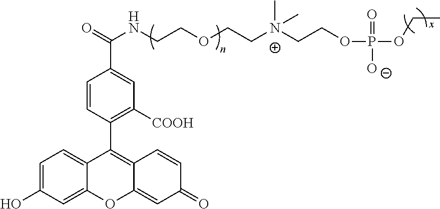

In some embodiments, the ProFL-PLE has the structure

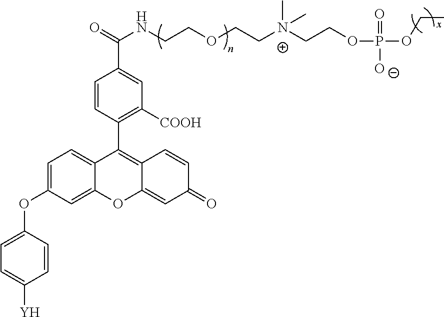

Some embodiments provided herein include aspects, which can prolong antiFL CART persistence by the use of or administration of unconjugated fluorescein, such as sodium fluorescein (NaFL) or disodium fluorescein or a salt of fluorescein. In some embodiments, cytotoxic events can be inhibited during CAR T cell therapy, by administration of or use of unconjugated fluorescein, such as NaFL, disodium fluorescein, or a salt of fluorescein. Without being bound to any one theory, unconjugated fluorescein may interact and bind with antiFL CAR T cells, whereupon the binding of the unconjugated fluorescein with the antiFL CAR can substantially reduce signaling of the CAR T cell. In some embodiments, the unconjugated fluorescein can be cleared by the patient, and the antiFL CAR T cells can bind and be activated by target FL-PLE, which is embedded in a cell membrane of a cancer cell in a tumor. In some embodiments, binding of unconjugated fluorescein increases the persistence of antiFL CAR T cells in a subject.

In some embodiments, NaFL can be administered at a concentration of 1 pM, 10 pM, 100 pM, 1 nM, 10 nM, 50 nM, 100 nM, 150 nM, 200 nM, 250 nM, 300 nM, 350 nM, 400 nM, 450 nM, 500 nM, 550 nM, 600 nM, 700 nM, 800 nM, 900 nM, 1 μM, 5 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 10 mM, 50 mM, 100 mM, 200 mM, 300 mM, 400 mM, 500 mM, 600 mM, 700 mM, 800 mM, 900 mM, 1 M, 2 M, 3 M, 4 M, 5 M, 10 M, or any concentration within a range defined by any two of the aforementioned concentrations.

In some embodiments, NaFL can be administered at an amount of 1 ng, 10 ng, 100 ng, 1 μg, 10 μg, 100 μg, 200 μg, 300 μg, 400 μg, 500 μg, 600 μg, 700 μg, 800 μg, 900 μg, 1 mg, 10 mg, 100 mg, 200 mg, 300 mg, 400 mg, 500 mg, 600 mg, 700 mg, 800 mg, 900 mg, 1 g, 2 g, 3 g, 4 g, 5 g, 6 g, 7 g, 8 g, 9 g, 10 g, 20 g, 30 g, 40 g, 50 g, 100 g, 1000 g, or any amount within a range defined by any two of the aforementioned amounts.

Some embodiments of the methods and compositions provided herein include aspects disclosed in Int. Pat. App. Pub. No. WO 2018/148224, and Int. Pat. App. Pub. No. WO 2019/156795, which are each hereby expressly incorporated by reference in their entireties.

Some embodiments of the methods and compositions provided herein include methods of treating or ameliorating or inhibiting a cancer in a subject. Some such embodiments include administering an effective amount to the subject a composition comprising, consisting essentially of, or consisting of a lipid conjugated to a target moiety; and administering a cell, such as a population of the cells, to the subject, wherein the cell comprises, consists essentially of, or consists of a chimeric antigen receptor (CAR) or T cell receptor (TCR), which specifically binds to the target moiety. Some embodiments include administering an unconjugated target moiety to the subject. In some such embodiments, administering an unconjugated target moiety to the subject can modulate the activity of the CAR T cells. For example, the target moiety can bind to the CAR T cell without invoking substantial signaling in the CAR T cell. In some embodiments, the presence of unconjugated target moiety can reduce effector function of the CAR T cell, compared to effector function in the absence of the unconjugated target moiety. In some embodiments, the presence of unconjugated target moiety can reduce undesirable side effects of the CAR T cell, such as cytokine release syndrome in a subject, compared to side effects in the absence of the unconjugated target moiety.

The term “% w/w” or “% wt/wt” as used herein refers to a percentage expressed in terms of the weight of the ingredient or agent over the total weight of the composition multiplied by 100. The term “% v/v” or “% vol/vol” as used herein refers to a percentage expressed in terms of the liquid volume of the compound, substance, ingredient, or agent over the total liquid volume of the composition multiplied by 100.

### Definitions

“Chimeric receptor” as used herein refers to a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain. Chimeric receptor can also be referred to as artificial T cell receptors, chimeric T cell receptors, chimeric immunoreceptors, and chimeric antigen receptors (CARs). These receptors can be used to graft the specificity of a monoclonal antibody or binding fragment thereof onto a T-cell, wherein transfer of their coding sequences is facilitated by viral vectors, such as a retroviral vector or a lentiviral vector. CARs are genetically engineered T-cell receptors designed to redirect T-cells to target cells that express specific cell-surface antigens. T-cells can be removed from a subject and modified so that they can express receptors that can be specific for an antigen by a process called adoptive cell transfer. The T-cells are reintroduced into the patient where they can then recognize and target an antigen. These CARs are engineered receptors that can graft a selected specificity onto an immune receptor cell. The term chimeric antigen receptors or “CARs” are also considered by some investigators to include the antibody or antibody fragment, such as a binding fragment of an antibody or scFv, the spacer, signaling domain, and transmembrane region. Due to the surprising effects of modifying the different components or domains of the CAR described herein, such as the epitope binding region (for example, antibody fragment, scFv, or portion thereof), spacer, transmembrane domain, and/or signaling domain), the components of the CAR are frequently distinguished throughout this disclosure in terms of independent elements.

“CAR T cell targeting agent,” (CTCT) is given its plain and ordinary meaning in view of the specification and can be described, for example as a composition that that can integrate into the membrane of a target cell. In the alternatives herein, the CTCT comprises, consists essentially of, or consists of a lipid, wherein the lipid comprises a target moiety and a masking moiety. In some embodiments, the masking moiety may be unmasked in the presence of low pH, ROS species and within a tumor microenvironment, for example. In some embodiments, the masking moiety may be unmasked by an enzyme or other protein. In some embodiments, the masking moiety inhibits specific binding of a CAR to the target moiety. The target moiety may be recognized and bound by a chimeric antigen receptor that is specific for the target moiety. In some alternatives herein, the masking moiety is removed at a pH of 4, 5, 6, or 6.5 or any pH in between a range defined by any two aforementioned values.

A “T cell receptor” or “TCR” is a molecule that is found on the surface of T lymphocytes or T cells that is responsible for the recognition of fragments of antigen bound to a major histocompatibility complex molecule.

“Target moiety” as described herein, refers to a specific group or site on a molecule or chemical that is a binding target for another chemical or protein of interest. In some alternatives, a complex is provided, wherein the complex comprises, consists essentially of, or consists of a chimeric antigen receptor (CAR) or a T cell receptor (TCR) joined to a lipid, wherein the lipid comprises a target moiety and the CAR is joined to said lipid through an interaction with said target moiety. In some alternatives, the target moiety is biotin, digoxigenin, dinitrophenol or fluorescein and the unconjugated haptens administered in the methods described herein can be salts of biotin, digoxigenin, dinitrophenol or fluorescein, such as sodium, disodium, or potassium salts of biotin, digoxigenin, dinitrophenol or fluorescein.

A “single-chain variable fragment,” (scFv) is a fusion protein that can have variable regions of the heavy (VH) and light chains (VL) of immunoglobulins, connected with a short linker peptide of ten to 25 amino acids. The short linker peptide can comprise 10, 11, 12, 13, 14, 15, 16, 17, 18, 19, 20, 21, 22, 23, 24, or 25 amino acids. The linker is usually rich in glycine for flexibility, as well as, serine or threonine for solubility, and can either connect the N-terminus of the VH with the C-terminus of the VL, or vice versa. This protein retains the specificity of the original immunoglobulin, despite removal of the constant regions and the introduction of the linker. The scFv can be specific for an antigen. “Antigen” or “Ag” as used herein, refers to a molecule that provokes an immune response. This immune response can involve either antibody production, or the activation of specific immunologically-competent cells, or both. An antigen can be generated, synthesized, produced recombinantly or can be derived from a biological sample. Such a biological sample can include, but is not limited to a tissue sample, a tumor sample, a cell or a biological fluid such, for example, blood, plasma or ascites fluid. In some alternatives herein, a composition is provided, wherein the composition comprises, consists essentially of, or consists of cells manufactured by any one of the alternative methods herein. In some alternatives, the cells comprise, consist essentially of, or consist of a chimeric antigen receptor, wherein the chimeric antigen receptor comprises, consists essentially of, or consists of a scFv that is specific for an antigen.

Some embodiments provided herein relate to a ScFv described herein as antiFL(FITC-E2 TyrH133A1a) (also referred to as antiFL(Try100gA1a), antiFL(FITC-E2 Mut2), or as antiFL(Mut2)), which can be incorporated into a CAR and used in a method in accordance with this disclosure (SEQ ID NO: 1), having an amino acid sequence of:

SVLTQPSSVSAAPGQKVTISCSGSTSNIGNNYVSWYQQHPGKAPKLMIYDV
SKRPSGVPDRFSGSKSGNSASLDISGLQSEDEADYYCAAWDDSLSEFLFGT
GTKLTVLGGGGGSGGGGSGGGGSQVQLVESGGNLVQPGGSLRLSCAASGFT
FGSFSMSWVRQAPGGGLEWVAGLSARSSLTHYADSVKGRFTISRDNAKNSV
YLQMNSLRVEDTAVYYCARRSYDSSGYWGHFASYMDVWGQGTLVTVS.

Some embodiments provided herein relate to a scFv described herein as antiFL(4M5.3), which can be incorporated into a CAR and used in a method in accordance with this disclosure (SEQ ID NO: 2), having an amino acid sequence of:

DVVMTQTPLSLPVSLGDQASISCRSSQSLVHSNGNTYLRWYLQKPGQSPKV
LIYKVSNRVSGVPDRFSGSGSGTDFTLKINRVEAEDLGVYFCSQSTHVPWT
FGGGTKLEIKSSADDAKKDAAKKDDAKKDDAKKDGGVKLDETGGGLVQPGG
AMKLSCVTSGFTFGHYWMNWVRQSPEKGLEWVAQFRNKPYNYETYYSDSVK
GRFTISRDDSKSSVYLQMNNLRVEDTGIYYCTGASYGMEYLGQGTSVTVS.

Some embodiments provided herein relate to a ScFv described herein as antiFL(4420), which can be incorporated into a CAR and used in a method in accordance with this disclosure (SEQ ID NO: 3), having an amino acid sequence of:

DVVMTQTPLSLPVSLGDQASISCRSSQSLVHSQGNTYLRWYLQKPGQSPKV
LIYKVSNRFSGVPDRFSGSGSGTDFTLKISRVEAEDLGVYFCSQSTHVPWT
FGGGTKLEIGGGGSGGGGSGGGGSEVKLDETGGGLVQPGRPMKLSCVASGF
TFSDYWMNWVRQSPEKGLEWVAQIRNKPYNYETYYSDSVKGRFTISRDDSK
SSVYLQMNNLRVEDMGIYYCTGSYYGMDYWGQGTSVTVSS.

Some embodiments provided herein relate to a ScFv described herein as antiFL(4D5Flu), which can be incorporated into a CAR and used in a method in accordance with this disclosure (SEQ ID NO: 4), having an amino acid sequence of:

DYKDIQMTQSPSSLSASVGDRVTITCRASQSLVHSQGNTYLRWYQQKPGKA
PKVLIYKVSNRFSGVPSRFSGSGSGTDFTLTISSLQPEDFATYYCQQSTHV
PWTFGQGTKVELKRAGGGGSGGGGSGGGGSSGGGSGGGGSGGGGSEVQLVE
SGGGLVQPGGSLRLSCAASGFTFSDYWMNWVRQAPGKGLEWVAQIRNKPYN
YETYYADSVKGRFTISRDTSKNTVYLQMNSLRAEDTAVYYCTGSYYGMDYW
GQGTLVTVSS.

Some embodiments provided herein relate to a ScFv described herein as antiFL(FITC-E2), which can be incorporated into a CAR and used in a method in accordance with this disclosure (SEQ ID NO: 5), having an amino acid sequence of:

SVLTQPSSVSAAPGQKVTISCSGSTSNIGNNYVSWYQQHPGKAPKLMIYDV
SKRPSGVPDRFSGSKSGNSASLDISGLQSEDEADYYCAAWDDSLSEFLFGT
GTKLTVLGGGGGSGGGGSGGGGSQVQLVESGGNLVQPGGSLRLSCAASGFT
FGSFSMSWVRQAPGGGLEWVAGLSARSSLTHYADSVKGRFTISRDNAKNSV
YLQMNSLRVEDTAVYYCARRSYDSSGYWGHFYSYMDVWGQGTLVTVS.

Some embodiments provided herein relate to a ScFv described herein as antiFL(FITC-E2 HisH131A1a), which can be incorporated into a CAR and used in a method in accordance with this disclosure (SEQ ID NO: 6), having an amino acid sequence of:

SVLTQPSSVSAAPGQKVTISCSGSTSNIGNNYVSWYQQHPGKAPKLMIYDV
SKRPSGVPDRFSGSKSGNSASLDISGLQSEDEADYYCAAWDDSLSEFLFGT
GTKLTVLGGGGGSGGGGSGGGGSQVQLVESGGNLVQPGGSLRLSCAASGFT
FGSFSMSWVRQAPGGGLEWVAGLSARSSLTHYADSVKGRFTISRDNAKNSV
YLQMNSLRVEDTAVYYCARRSYDSSGYWGAFYSYMDVWGQGTLVTVS.

“Antigen specific binding domains” can include protein or protein domains that can specifically bind to an epitope on a protein at a low or high binding affinity (fM to mM binding capacity). In some alternatives, the fusion protein comprises, consists essentially of, or consists of a protein or portion thereof that can modulate an immune response. In some alternatives, the protein comprises, consists essentially of, or consists of an antigen specific binding domain.

T-cells” or “T lymphocytes” as used herein, can be from any mammalian species, preferably primate, including monkeys, dogs, or humans. In some alternatives the T-cells are allogeneic (from the same species but different donor) as the recipient subject; in some alternatives the T-cells are autologous (the donor and the recipient are the same); in some alternatives the T-cells are syngeneic (the donor and the recipients are different but are identical twins).

“Individual”, “subject” or “patient,” as described herein, refers to any organism upon which the alternatives described herein may be used or administered, e.g., for experimental, diagnostic, prophylactic, and/or therapeutic purposes. Subjects or patients include, for example, animals. In some alternatives, the subject is mice, rats, rabbits, non-human primates, or humans. In some alternatives, the subject is a cow, sheep, pig, horse, dog, cat, primate or a human.

Some embodiments relate to a CAR T cell tumor targeting (CTCT) agent. Some embodiments provided herein relate to a phospholipid ether (PLE) tethered to the hapten fluorescein (FL-PLE). “Fluorescein” as described herein, is a synthetic organic compound that is soluble in water and alcohol. It is widely used as a fluorescent tracer for many applications. In the alternatives herein, fluorescein is a target moiety on a lipid that is specifically recognized by a chimeric antigen receptor designed and/or selected for its ability to bind or interact with the fluorescein. In some alternatives, the lipid is a phospholipid ether.

“Hapten” as described herein is a small molecule that elicit an immune response only when conjugated or attached to a large carrier such as a protein. In some embodiments, the carrier may be one that also does not elicit an immune response by itself. In other embodiments, the carrier may be one that does elicit an immune response by itself. Once the body has generated antibodies to a hapten-carrier adduct, the small-molecule hapten may also be able to bind to the antibody, but it will usually not initiate an immune response; usually only the hapten-carrier adduct can do this. In some embodiments, a hapten is a small molecule binding moiety, which can be bound by or have specificity towards a scFv or antibody.

“Cancer,” as described herein, is a group of diseases involving abnormal cell growth with the potential to invade or spread to other parts of the body. Subjects that can be addressed using the methods described herein include subjects identified or selected as having cancer, including but not limited to colon, lung, liver, breast, renal, prostate, ovarian, skin (including melanoma), bone, leukemia, multiple myeloma, or brain cancer, etc. Such identification and/or selection can be made by clinical or diagnostic evaluation. In some alternatives, the tumor associated antigens or molecules are known, such as melanoma, breast cancer, brain cancer, squamous cell carcinoma, colon cancer, leukemia, myeloma, or prostate cancer. Examples include but are not limited to B cell lymphoma, breast cancer, brain cancer, prostate cancer, and/or leukemia. In some alternatives, one or more oncogenic polypeptides are associated with kidney, uterine, colon, lung, liver, breast, renal, prostate, ovarian, skin (including melanoma), bone, brain cancer, adenocarcinoma, pancreatic cancer, chronic myelogenous leukemia or leukemia. In some alternatives, a method of treating, ameliorating, or inhibiting a cancer in a subject is provided by administering one or more of the CARs described herein to a subject in need thereof. In some alternatives, the cancer is breast, ovarian, lung, pancreatic, prostate, melanoma, renal, pancreatic, glioblastoma, neuroblastoma, medulloblastoma, sarcoma, liver, colon, skin (including melanoma), bone or brain cancer. In some alternatives, the subject that receives one of the therapies described herein is also selected to receive an additional cancer therapy, which can include surgery, a cancer therapeutic, radiation, chemotherapy, targeted therapy, immunotherapy, hormonal therapy, or a cancer therapy drug. In some alternatives, the cancer therapy drug provided is, comprises, consists essentially of, or consists of Abiraterone, Alemtuzumab, Anastrozole, Aprepitant, Arsenic trioxide, Atezolizumab, Azacitidine, Bevacizumab, Bleomycin, Bortezomib, Cabazitaxel, Capecitabine, Carboplatin, Cetuximab, Chemotherapy drug combinations, Cisplatin, Crizotinib, Cyclophosphamide, Cytarabine, Denosumab, Docetaxel, Doxorubicin, Eribulin, Erlotinib, Etoposide, Everolimus, Exemestane, Filgrastim, Fluorouracil, Fulvestrant, Gemcitabine, Imatinib, Imiquimod, Ipilimumab, Ixabepilone, Lapatinib, Lenalidomide, Letrozole, Leuprolide, Mesna, Methotrexate, Nivolumab, Oxaliplatin, Paclitaxel, Palonosetron, Pembrolizumab, Pemetrexed, Prednisone, Radium-223, Rituximab, Sipuleucel-T, Sorafenib, Sunitinib, Talc Intrapleural, Tamoxifen, Temozolomide, Temsirolimus, Thalidomide, Trastuzumab, Vinorelbine or Zoledronic acid.

“Tumor microenvironment” as described herein is a cellular environment, wherein a tumor exists. Without being limiting, the tumor microenvironment can include surrounding blood vessels, immune cells, fibroblasts, bone marrow-derived inflammatory cells, lymphocytes, signaling molecules or the extracellular matrix (ECM).

“Cytokine release syndrome” (CRS), or “cytokine storm” as described herein refers to an uncontrolled release of proinflammatory cytokines by immune cells, including T cells, natural killer cells, macrophages, dendritic cells, B cells, monocytes, neutrophils, leukocytes, lymphocytes, in response to a disease, infection, or immunotherapy. Diseases or infections that can cause CRS include but are not limited to bacterial infections, viral infections, graft-versus-host disease, cytomegalovirus, Epstein-Barr virus, streptococcus, Pseudomonas, influenza, H5N1, H1N1, variola virus, coronavirus, SARS, sepsis, or lipopolysaccharide. Immunotherapies that can cause CRS include but are not limited to rituximab, obinutuzumab, alemtuzumab, brentuximab, dacetuzumab, nivolumab, theralizumab, oxaliplatin, lenalidomide, or CAR T therapy. CRS can be treated using anti-inflammatory therapies, including but not limited to anti-cytokine antibodies, angiotensin-converting enzyme inhibitors, angiotensin II receptor blockers, corticosteroids, free radical scavengers, or TNF-α blockers.

As used herein, the term “cytokine” refers to small proteins, polypeptides, or peptides that are involved in inflammatory signaling. Cytokines include but are not limited to chemokines, interferons, interleukins, lymphokines, tumor necrosis factors, CCL1, CC12, CCL3, CCL4, CCL5, CCL6, CCL7, CCL8, CCL9, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CX3CL1, XCL1, XCL2, INFα, INFβ, INFγ, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, IL-37, IL-38, GM-CSF, TNFα, TNFβ, TNFγ, TNFSF4, TNFSF5, TNFSF6, TNFSF7, TNFSF8, TNFSF9, TNFSF10, TNFSF11, TNFSF12, TNFSF13, TNFSF13B, TNFSF14, TNFSF15, TNFSF18, or TNFSF19, or any combination thereof.

### Methods of Therapy

Some embodiments of the methods and compositions provided herein include methods of treating or ameliorating or inhibiting a cancer in a subject. Some such embodiments include administering an effective amount to the subject a composition comprising, consisting essentially of, or consisting of a lipid conjugated to a target moiety; and administering a cell, such as a population of the cells, to the subject, wherein the cell comprises, consists essentially of, or consists of a chimeric antigen receptor (CAR) or T cell receptor (TCR), which specifically binds to the target moiety. Some embodiments include administering an unconjugated target moiety to the subject, such as a salt of biotin, digoxigenin, dinitrophenol or fluorescein, such as sodium, disodium, or potassium salts of biotin, digoxigenin, dinitrophenol or fluorescein. In some such embodiments, administering an unconjugated target moiety to the subject can modulate the activity of the CART cells. For example, the target moiety can bind to the CAR T cell without invoking substantial signaling in the CAR T cell. In some embodiments, the presence of unconjugated target moiety can reduce effector function of the CAR T cell. In some embodiments, the presence of unconjugated target moiety can reduce undesirable side effects of the CAR T cell, such as cytokine release syndrome in a subject.

The terms “treating,” “treatment,” “therapeutic,” or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

Some embodiments described herein relate to a method of treating, inhibiting, ameliorating, preventing, or slowing a disease or disorder described herein. In some embodiments, the methods include administering to a subject identified as suffering from the disease or disorder described herein an effective amount of a cell described herein, or a pharmaceutical composition that includes an effective amount of a cell as described herein. In some embodiments, the cell comprises, consists essentially of, or consists of a chimeric antigen receptor (CAR) or T cell receptor (TCR), which specifically binds to a target moiety. In some embodiments, the method further includes administering to the subject an effective amount of the unconjugated target moiety. In some embodiments, the administration of the effective amount of the unconjugated target moiety reduces effector function or cytokine production of the cell comprising, consisting essentially of, or consisting of the CAR or TCR. Other embodiments described herein relate to using a cell or target moiety as described herein in the manufacture of a medicament for treating, inhibiting ameliorating, preventing, or slowing the disease or disorder described herein. Still other embodiments described herein relate to the use of a cell or target moiety as described herein or a pharmaceutical composition that includes an effective amount of a cell or target moiety as described herein for treating, inhibiting ameliorating, preventing, or slowing the disease or disorder described herein.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1—In Vitro antiFL CAR T Cell Recognition and Activation Through FL-PLE and Nonrecognition and Activation in Presence of Free Fluorescein Molecules

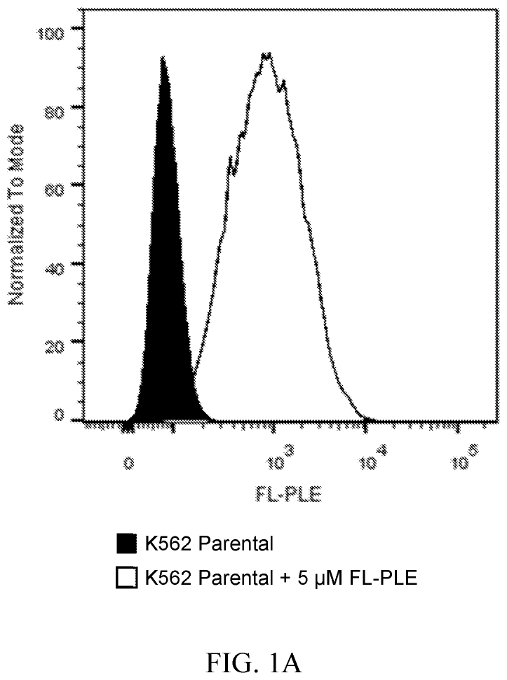

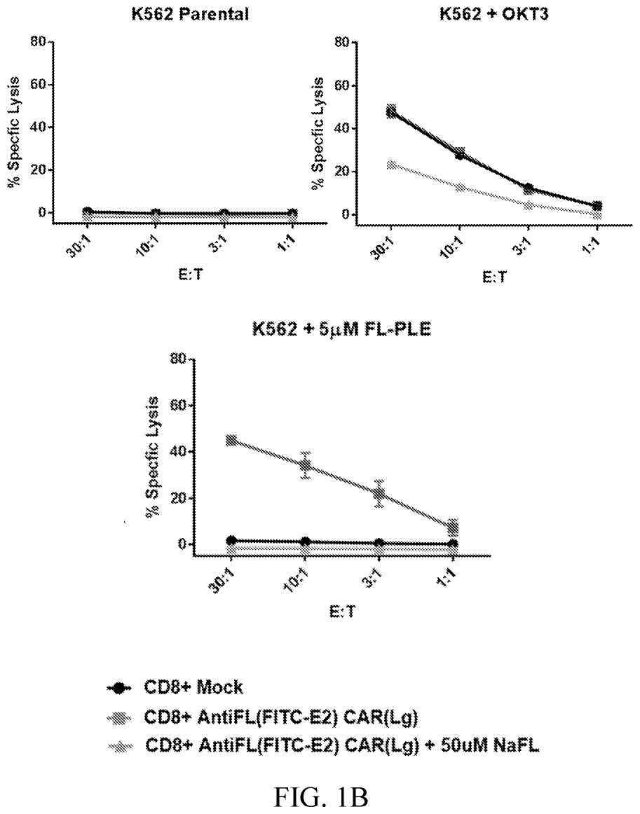

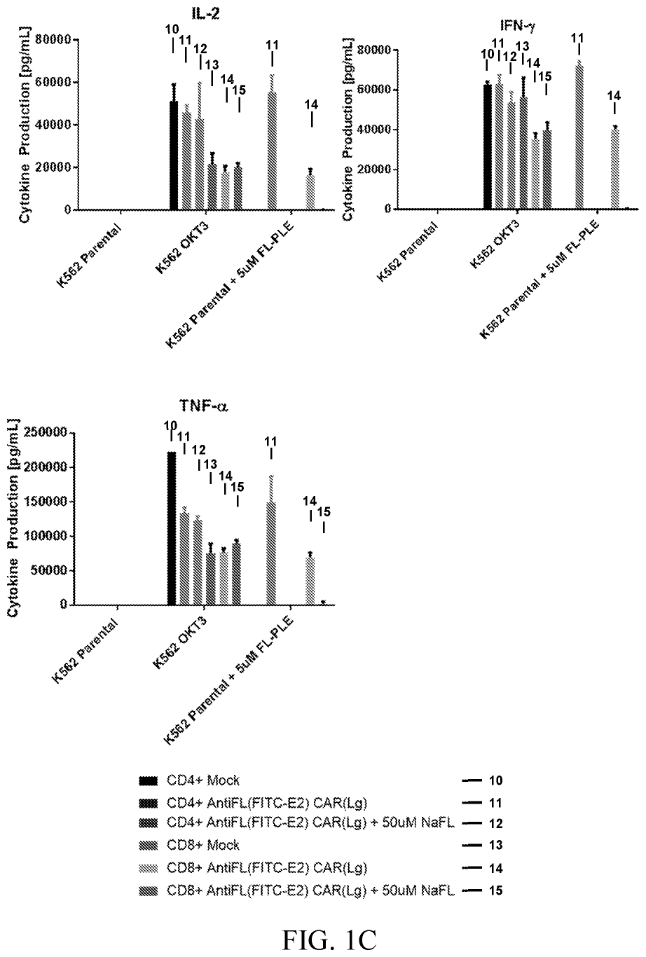

K562 (leukemia) cells were incubated with FL-PLE overnight. Cell integration of FL-PLE was analyzed by flow cytometry (FIG. 1A). There was a clear shift from the control K562 parental with the K562 parental incubated with 5 μM FL-PLE. This slight shift corresponded to a difference in the amount of FL exposed on the surface of the cell for CAR T cell recognition. These cells were used in a chromium release assay (FIG. 1B) and a cytokine release assay (FIG. 1C) to test whether the activation of CD8+ and CD4+ antiFL(FITC-E2) CAR T cells through FL-PLE integrated into K562 cells could be stopped in the presence of sodium fluorescein (NaFL). The K562 OKT3+ cells, a model cell line, were able to confirm the endogenous activation of T cells through the TCR of both antiFL(FITC-E2) CAR T cell and Mock in all conditions. From these experiments, antiFL(FITC-E2) CAR T cells recognized the FL moiety of the FL-PLE integrated into the plasma membrane and were activated. However, the presence of 50 μM NaFL was able to stop the recognition and activation of antiFL(FITC-E2) CAR T cells.

### Example 2—In Vivo Intratumoral Use of Sodium Fluorescein (NaFL) to Reverse Cytotoxic Episodes by antiFL CAR T Cells

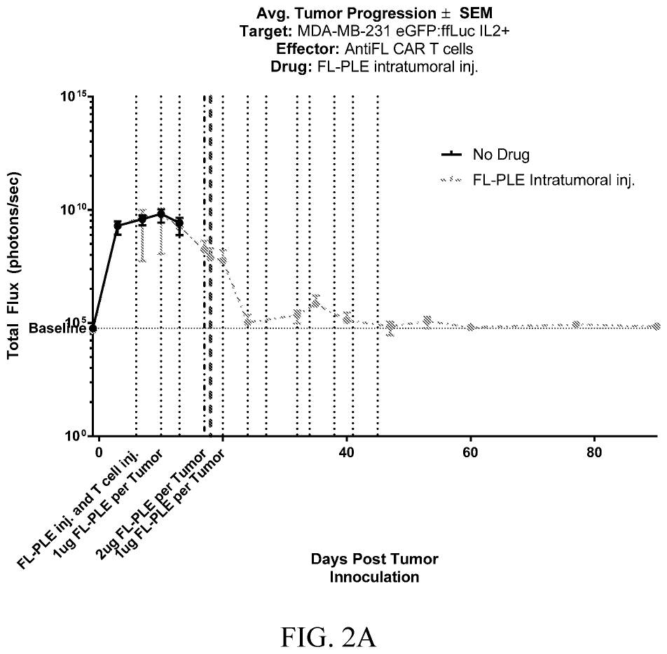

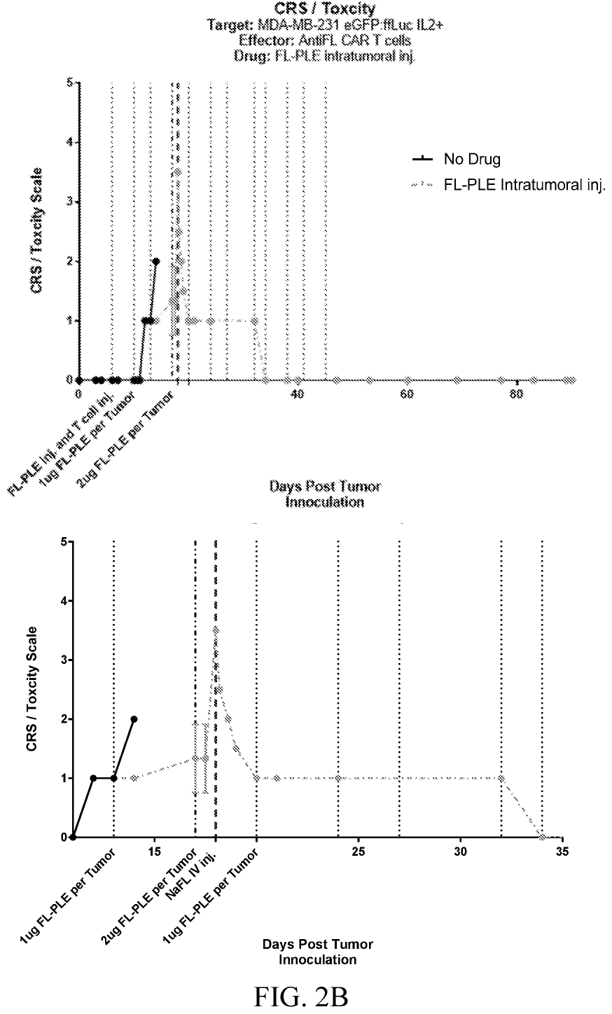

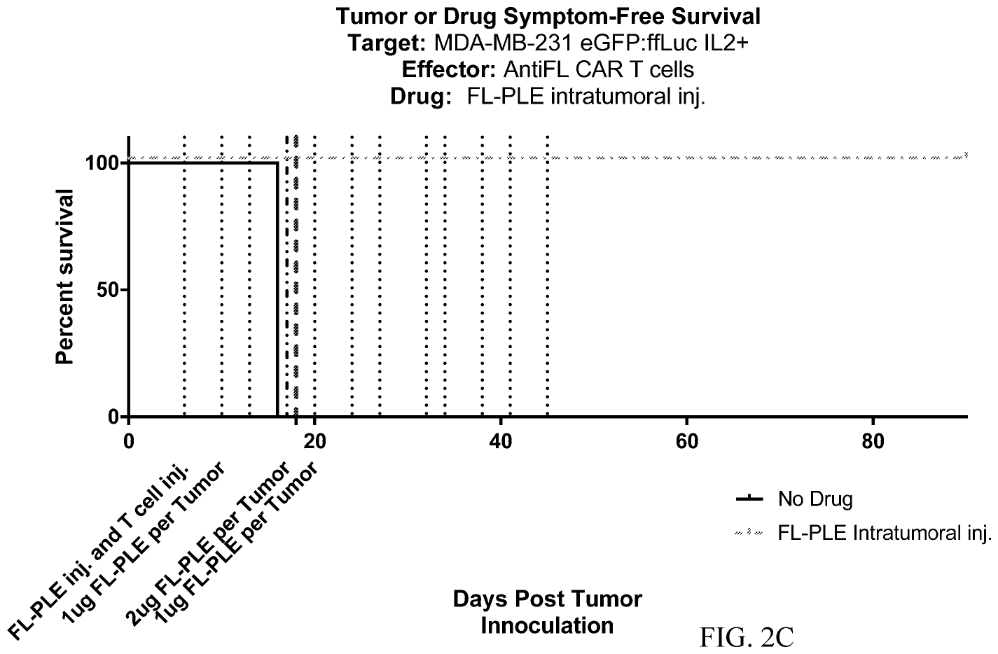

After an adenocarcinoma (MDA-MB-231) tumor was established in 2 groups of mice by subcutaneous injection (2 tumors per mouse in opposite flanks), the mice received an intravenous injection of antiFL(FITC-E2) CART cells on day 6. The control group (no drug) only received the antiFL(FITC-E2) CAR T cells and the tumor progressed as normal. The second group received intratumoral injection of 1 μg FL-PLE prior to T cell injection on day 6 followed by re-dosing twice a week with FL-PLE until the tumor had regressed on day 45 (FIG. 2A). The mice were monitored for cytokine release syndrome (CRS) and cytotoxicity on a scale ranging from 1 (healthy) to 5 (dead) (FIG. 2B). On day 17, the intratumoral dose of FL-PLE was increased to 2 μg per tumor. On day 18, the mice had levels of CRS/toxicity between 3 and 4 at which point they received a dose of 0.56 mg of NaFL via an intravenous injection. Within minutes, the cytotoxicity level had dropped and by 12 hours had return to roughly the baseline levels prior to the past injection. After this, all doses of FL-PLE were administered at 1 μg per tumor. Every mouse from this group lived to day 90 (FIG. 2C). Also, at day 90 there were no tumors present, as seen in FIG. 2A. This experiment demonstrated that NaFL quickly reduced cytotoxic events and that following administration and clearance of NaFL, the antiFL CAR T cells continued to function.

### Example 3—In Vitro Use of NaFL is Dose Titratable in Reducing the Amount of Activation of an antiFL CAR T Cell

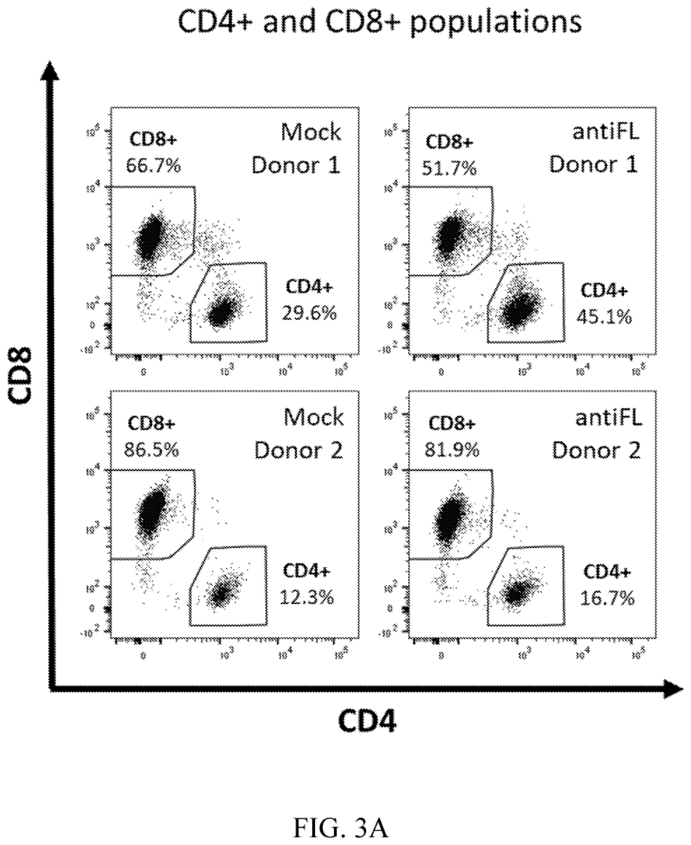

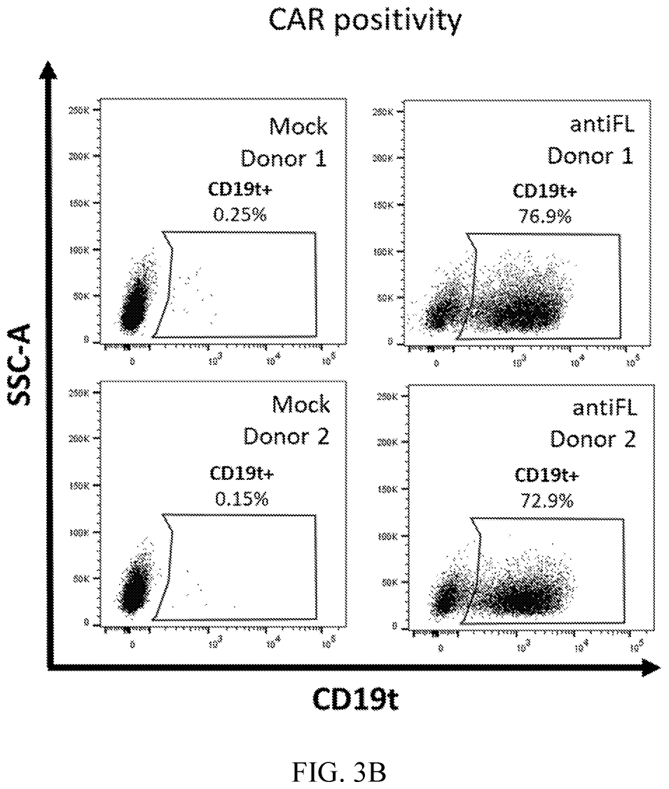

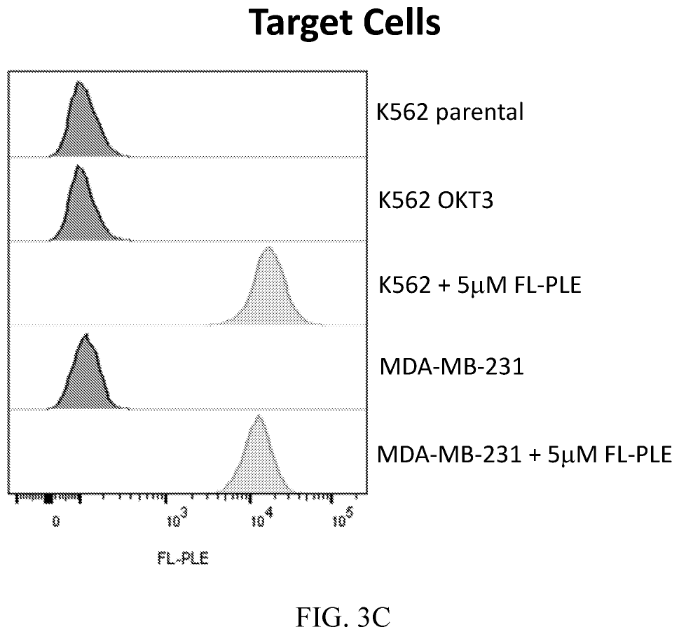

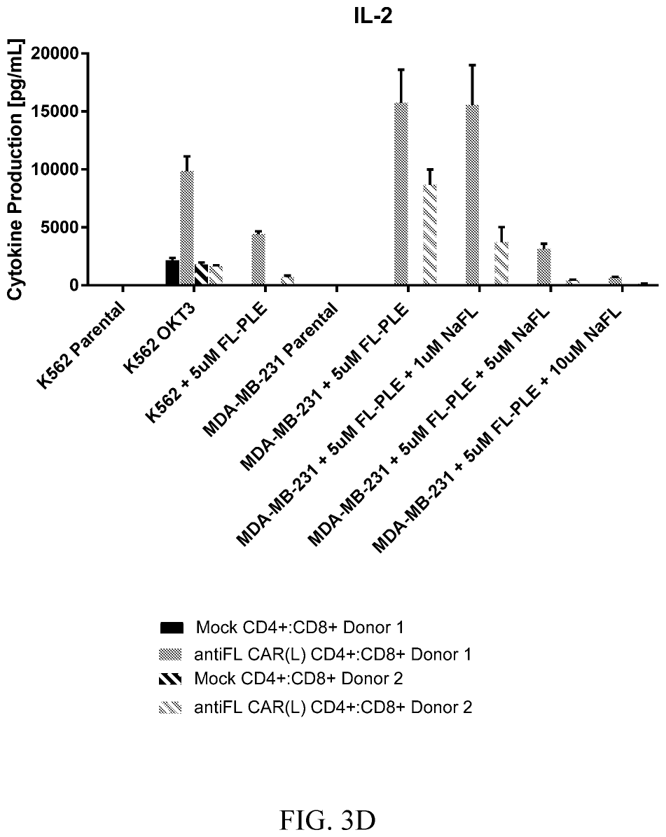

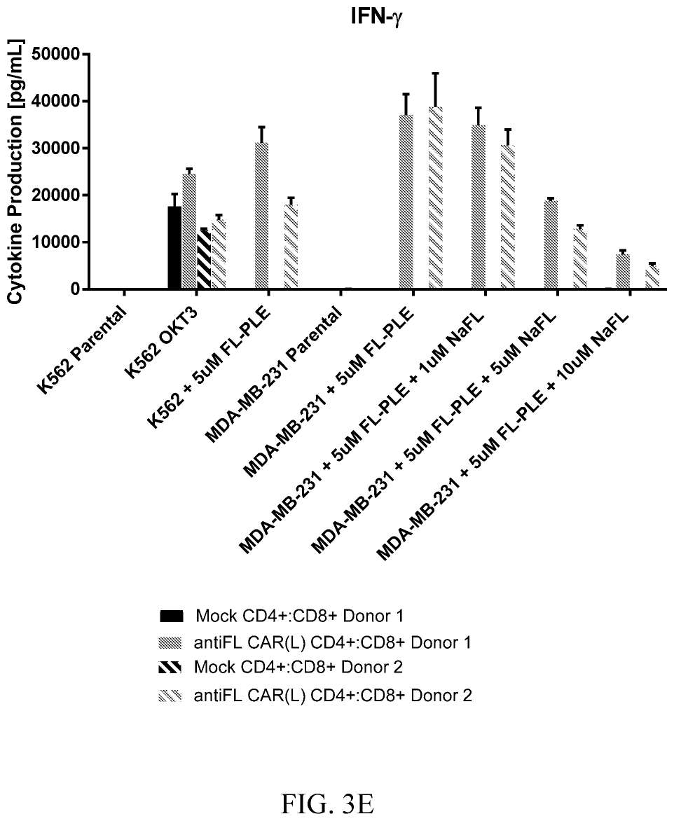

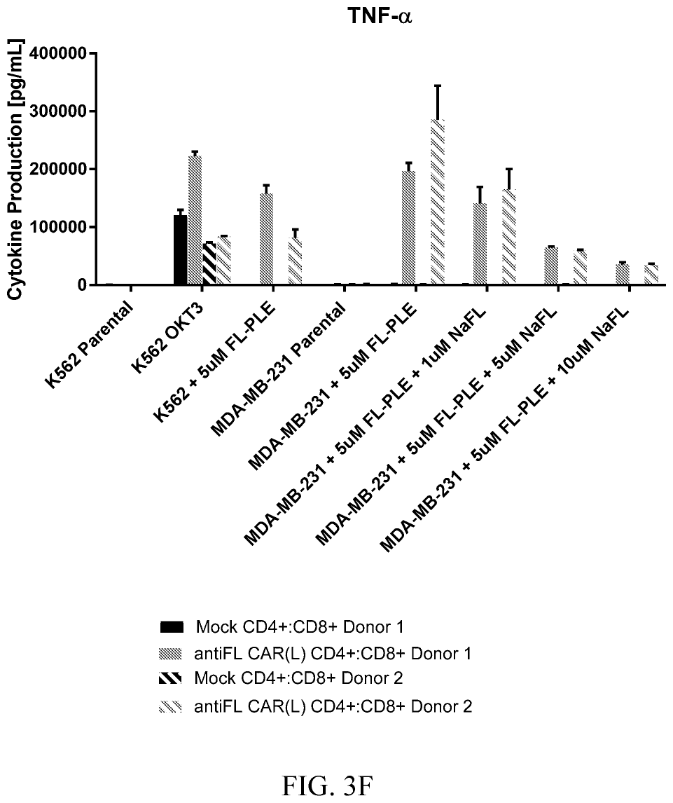

CD4+ and CD8+ T cells from peripheral blood mononuclear cell preparations from two donors were lentivirally transduced to express long CAR cassettes comprising the antiFL(Mut2) CAR. The CAR cassette also contains the gene for a double mutant dihydrofolate reductase that allows for methotrexate positive selection to enrich for transduced CAR-expressing cells as well as the gene for the truncated CD19 (CD19t) surface marker that denotes CAR positivity. The selected cells are then subjected to a standard rapid expansion protocol using irradiated TM-LCL and PBMC feeder cells. After 14 days of expansion, cells were stained with anti-CD4, anti-CD8, and anti-CD19 antibodies as well as a live-dead stain to characterize surface phenotypes by flow cytometry. Flow plots for the CD4 and CD8 populations (FIG. 3A) and CAR positivity (FIG. 3B) are shown for mock and antiFL(Mut2) CAR T cells for both donors. These are the effector cells to be used in subsequent experiments.

K562 parental (negative control), K562 OKT3+(T cell activating positive control), and MDA-MB-231 target cells were incubated with 5 μM FL-PLE in PBS for 30 minutes followed by a wash to remove unbound FL-PLE. Cells were then returned to complete media. Cell integration of FL-PLE was analyzed by flow cytometry (FIG. 3C). There is a clear shift in FL-PLE amounts from the parental cell lines in contrast to those incubated with 5 μM FL-PLE. The amount of shift corresponds to the amount of fluorescein exposed on the surface of the cell (which is important for antiFL CAR T cell recognition). K562 OKT3+ cells, which were generated as a positive control to test the endogenous activation of T cells through the TCR) match the parental K562 line, showing that the addition of OKT3 does not interfere with fluorescein detection. These target cells are used for the following cytokine release assay.

A cytokine release assay was set up to examine how a dose titration of NaFL (0, 1, 5, and 10 μM) affects the activation of antiFL CAR T cells. The CAR T cells are the antiFL(Mut2) CAR T cells generated in this example. Target cells were incubated with FL-PLE/NaFL for 24 hours or overnight. The media supernatant from target cell cultures were harvested and constituent cytokines were measured (e.g. with a Bio-Plex Human Cytokine Screening Panel). The cytokines IL-2 (FIG. 3D), IFN-γ (FIG. 3E), and TNF-α (FIG. 3F) were measured. The antiFL(Mut2) CAR T cells are able to recognize the fluorescein moiety of the FL-PLE integrated in the plasma membrane of the target cells and are able to activate with both the K562 and MDA-MB-231 cells loaded with FL-PLE whereas the mock T cells do not activate in the presence of these cells. The amount of activation is decreased by the amount of NaFL present in the solution in a linear fashion for all three cytokines tested for both donor T cells. Therefore, NaFL can be used in vitro to reduce cytokine production by antiFL CAR T cells in a dose titratable manner.

### Example 4—In Vivo Intravenous Use of NaFL to Reverse Cytotoxic Episodes by antiFL CAR T Cells

Mice were prepared to test whether intravenous (IV) administration of NaFL as opposed to intratumoral administration (as seen in Example 2) can be used to reduce cytokine production by antiFL CAR T cells.

Group A—control (5 mice): MDA-MB-231 tumor+antiFL(Mut2) CAR T cells.

Group B—FL-PLE without NaFL (5 mice): MDA-MB-231 tumor+antiFL(Mut2) CAR T cells+1 mg IV FL-PLE.

Group C—FL-PLE and NaFL (5 mice): MDA-MB-231 tumor+antiFL(Mut2) CAR T cells+1 mg IV FL-PLE+0.5 mg IV NaFL (as needed).

Group D—No tumor control (3 mice): antiFL(Mut2) CAR T cells+1 mg IV FL-PLE+0.5 mg IV NaFL (as needed).

Mice receiving FL-PLE (Groups B, C, and D) were administered FL-PLE IV first. AntiFL(Mut2) CAR T cells were subsequently administered in all mice 2 days following FL-PLE administration. No cytokine toxicity were observed in any mice over a 2 day period following CAR T administration. Following this 2 day period, a second injection of FL-PLE was administered in Groups B-D. After 24 hours or overnight, cytokine toxicity symptoms in mice of Groups B-D was observed. Reduction of cytokine toxicity symptoms in mice of Groups C and D was observed within 15 minutes after administration of NaFL. After 2 days following the first administration of NaFL, optionally with additional IV injections of NaFL, mice of Groups C and D had cytokine levels comparable to baseline. Mice in Group B did not recover from the cytokine toxicity symptoms and were euthanized. This demonstrates that NaFL is well tolerated in vivo and can be used to reduce the cytokine response caused by antiFL CAR T cells by intravenous administration even if the subject does not have a tumor.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of treating, ameliorating, inhibiting, or providing a therapy to a subject having a cancer, comprising:
(a) administering to the subject a composition comprising a lipid conjugated to a target moiety, such as biotin, digoxigenin, dinitrophenol or fluorescein;
(b) administering a cell to the subject, wherein the cell comprises a chimeric antigen receptor (CAR) or T cell receptor (TCR), which specifically binds to the target moiety; and
(c) administering the target moiety unconjugated to the lipid to the subject, such as a salt of biotin, digoxigenin, dinitrophenol or fluorescein, e.g., sodium, disodium, or a potassium salt of biotin, digoxigenin, dinitrophenol or fluorescein.

2-17. (canceled)


SEQUENCE LISTING (6 sequences)

SEQ ID NO: 1 - protein, 251 aa, Artificial Sequence
  antiFL(FITC-E2 Mut2) CAR
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFASYMDVWGQGTLVTVS

SEQ ID NO: 2 - protein, 254 aa, Artificial Sequence
  antiFL(4M5.3) CAR
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 3 - protein, 244 aa, Artificial Sequence
  antiFL(4420) CAR
DVVMTQTPLSLPVSLGDQASISCRSSQSLVHSQGNTYLRWYLQKPGQSPKVLIYKVSNRFSGVPDRFSGSGSGTDFTLKISRVEAEDLGVYFCSQSTHVPWTFGGGTKLEIGGGGSGGGGSGGGGSEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 4 - protein, 265 aa, Artificial Sequence
  antiFL(4D5Flu) CAR
DYKDIQMTQSPSSLSASVGDRVTITCRASQSLVHSQGNTYLRWYQQKPGKAPKVLIYKVSNRFSGVPSRFSGSGSGTDFTLTISSLQPEDFATYYCQQSTHVPWTFGQGTKVELKRAGGGGSGGGGSGGGGSSGGGSGGGGSGGGGSEVQLVESGGGLVQPGGSLRLSCAASGFTFSDYWMNWVRQAPGKGLEWVAQIRNKPYNYETYYADSVKGRFTISRDTSKNTVYLQMNSLRAEDTAVYYCTGSYYGMDYWGQGTLVTVSS

SEQ ID NO: 5 - protein, 251 aa, Artificial Sequence
  antiFL(FITCE2) CAR
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVS

SEQ ID NO: 6 - protein, 251 aa, Artificial Sequence
  antiFL(FITCE2 HisH131Ala) CAR
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGAFYSYMDVWGQGTLVTVS